(19) United States
(12) Patent Application Publication
(10) Pub. No.: US 2023/0074695 A1
(43) Pub. Date: Mar. 9, 2023

(54) METHOD FOR PRODUCING A NONWOVEN ITEM, NONWOVEN ITEM AND HYGIENE ARTICLE

(71) Applicant: Mondi AG, Wien (AT)

(72) Inventors: Jan Michael TRINKAUS, Euskirchen (DE); Christian BERNDT, Aschersleben (DE); Patricia RODRIGUEZ, Aschersleben (DE); Marko FLECK, Aschersleben (DE); Frank MUELLER, Quedlinburg (DE)

(73) Assignee: Mondi AG, Wien (AT)

(21) Appl. No.: 17/891,277

(22) Filed: Aug. 19, 2022

(30) Foreign Application Priority Data
Aug. 20, 2021 (EP) 21 192 332.1

(51) Int. Cl. D04H 1/541; D04H 1/544; D04H 1/485; D04H 3/007

(52) U.S. Cl. CPC D04H 1/5412; D04H 1/544; D04H 1/485; D04H 3/007; D10B 2321/02; D10B 2509/00; D10B 2201/02; D10B 2201/04; D10B 2201/22

(57) ABSTRACT

A method for producing a nonwoven element particularly for hygiene products, has at least the following steps:
forming a fibrous web sheet with a width direction extending transverse to the production direction and a thickness direction perpendicular thereto by supplying staple fibers from at least a first group which are formed from a thermoplastic material,
consolidating the fibrous web sheet to form a nonwoven web by heating exclusively a first side of the fibrous web sheet through contact with a heated surface such that the staple fibers of the first group are partially melted, and
cooling the nonwoven web.

### CROSS-REFERENCE TO RELATED APPLICATIONS

This application claims priority under 35 USC 119 of European Application No. EP 21 192 332.1, filed on Aug. 20, 2021, the disclosure of which is herein incorporated by reference.

### BACKGROUND OF THE INVENTION

The present invention is directed to a method for producing a nonwoven element, particularly for hygiene products, e.g., diapers, hygienic wet wipes or incontinence articles.

Nonwovens are also commonly referred to as bonded fiber fabrics and denote an initially loose, randomly ordered fiber composite which is subsequently consolidated. The loose or at least slightly consolidated fiber composite is also commonly referred to as fibrous web. Various methods are known for consolidating the fibrous web to form a nonwoven based on a physical-mechanical interlacing of the individual fibers and/or on a chemical or physical-thermal gluing of the fibers.

Such nonwovens are suitable for many fields of application, but it will be assumed for purposes of the invention that the nonwoven elements in particular constitute part of hygiene products. For example, the outer layer of diapers have such a nonwoven element in order to impart a textile character to the outer side. For this purpose, the nonwoven element is generally attached to a film. This outer layer is also commonly referred to as a “backsheet” and serves to receive the absorbent core. Further, this backsheet also outwardly seals the absorbent core. When used for this purpose, the nonwoven elements are usually formed from spun fibers (spun nonwovens) which are spun as continuous filaments from a melt or solution and are subsequently stretched in longitudinal direction. Hydroentangled (spun-laced) fibers can also be used in this respect.

Nowadays, there is an increasing demand to also use natural fibers or other non-fusible fibers in such nonwoven elements. However, such fibers cannot readily be used in combination with spun fibers because they are not continuous filaments formed in a joint production process. Fibers which—in contrast to spun fibers—are not formed from continuous filaments and accordingly have a limited length are also commonly referred to as staple fibers which are often laid down as carded webs (carded nonwoven). In contrast to nonwovens formed of spun fibers, both stiffness and tensile strength are substantially reduced. Therefore, in many cases staple fibers are not suitable for use in hygiene products and particularly not as part of an outer layer of a diaper.

Further, such nonwovens can also be used as hook-and-loop fasteners in combination with velcro hooks. A hook and loop fastener of this kind is contemplated, for example, for closing diapers. In this case, the nonwoven elements form the so-called “landing zone” in which little gripping hooks mechanically engage. A substantial advantage of hook-and-loop fasteners is based on the fact that they can be repeatedly unfastened and re-fastened. In this respect, the holding properties are not contingent on possible soiling contact with personal care products such as creams or baby oil or other liquids. In order to ensure a lasting holding property, such hook-and-loop fasteners are usually formed from knitted or woven textiles to ensure that the individual hooks have sufficient engagement surfaces.

If nonwovens are to be used as landing zone, spun fibers are usually also used in this case. Owing to their long length, such fibers cannot easily be pulled out when unfastening the hook-and-loop fastener and therefore also do not form a sufficient engagement surface for the hooks after repeated opening.

For this reason, repeated attempts have been undertaken in the past to combine the strength of a nonwoven formed of spun fibers with the flexibility and softness of staple fibers. For example, EP 3 058 127 B1 describes a nonwoven element in which a first ply of continuous filaments and a second ply of puckered staple fibers are joined together by means of a high-pressure water jet. Accordingly, a nonwoven element is produced from spun fibers on the one hand and a nonwoven element is produced from staple fibers on the other hand, and the individual nonwoven elements are subsequently connected to one another as layers by means of a separate consolidation process. The resulting nonwoven element accordingly has different characteristics on different sides, and tensile strength and stiffness are decisively ensured via the spun fibers and the softness and flexibility for integrating different fiber materials are ensured by the layer of staple fibers.

Although such a method has basically proven itself, it nevertheless requires a relatively large expenditure on production because two different production processes and therefore also different machines must be provided for production.

### SUMMARY OF THE INVENTION

In view of the foregoing, the present invention has the object of showing a method for producing a nonwoven element which can be implemented in a substantially simpler and more cost-effective way compared to previous solutions and by means of which a nonwoven element can be produced that is distinguished by a high degree of flexibility with respect to the integration of different types of fiber.

This object is met by a method for producing the nonwoven element that comprises at least the following steps:
forming a fibrous web sheet with a width direction extending transverse to the production direction and a thickness direction perpendicular to the width direction by supplying staple fibers from at least a first group which are formed from a thermoplastic material, particularly a thermoplastic plastic,
consolidating the fibrous web sheet to form a nonwoven web by heating exclusively one side of the fibrous web sheet through contact with a heated surface such that the staple fibers of the first group are partially melted, and
cooling the nonwoven web.

Correspondingly, a nonwoven web comprising exclusively staple fibers is formed, and the use of additional machinery for producing spun fibers is dispensed with. Further, additional reinforcement by means of hydroentanglement and subsequent drying is also not required. With this in mind, therefore, different types of staple fibers can be used, and at least a first group of staple fibers is provided which comprise a thermoplastic material and accordingly partially melt when coming in contact with the heated surface. In this regard, partial melting refers to the fact that the heat required for this takes place exclusively on one side through contact with the heated surface so that the fibers of the first group are melted proceeding from the first side and the melt zone does not extend over the entire thickness direction. Accordingly, the second side of the nonwoven web is not in contact with a heated surface.

Further, the heated surface forms a certain pressure on the fibrous web sheet proceeding from the first side during the heating so that the melting fibers are compressed to a certain extent and consolidate after cooling. This creates a kind of lattice structure with an inhomogeneous distribution of orifices via which the tensile strength of the nonwoven is increased but the material characteristics change along the thickness direction. Such consolidation can essentially be compared to an ironing process because the heat is introduced exclusively on one side into the fibrous web sheet accompanied by pressure. Accordingly, the surface is preferably smooth with no textures.

On the whole, a nonwoven element having different haptic properties on both sides due to its asymmetrical treatment is formed by the inventive method. Accordingly, in addition to the consolidation, a kind of smoothing takes place so that the side of the fibrous web sheet or nonwoven web that is not consolidated is substantially softer and fluffier than the consolidated first side. In view of this, this consolidation process is also referred to within the framework of the invention as thermal smoothing. Therefore, the consolidated side is particularly suited for attaching the nonwoven element to substrates, e.g., a film, while the side that is not consolidated offers the user a pleasant haptic feel.

It will be appreciated that such a method assumes that the melting temperature of the staple fibers of the first group is lower than or at least equal to the heating temperature of the heated surface. At the same time, the heated surface should also have a sufficiently high heating capacity so that the fibrous web sheet guided along the heated surface is not exposed to fluctuations in temperature on the heated surface, which could change the material characteristics in production direction.

According to a preferred embodiment of the invention, the heated surface can be part of a heating roll which is formed as a guide roll and over which the fibrous web sheet is guided. Accordingly, the fibrous web sheet wraps around the heating roll at a certain angle, referred to hereinafter as wrap angle. This wrap angle and the velocity of the fibrous web sheet in production direction define the relevant period of contact between the fibrous web sheet and the heated surface. This contact time together with the heating temperature of the surface, the pressure between the fibrous web sheet and the heated surface, and the sheet tensioning are decisive for the manner in which the consolidation is carried out in the course of thermal smoothing. In principle, the heated surface can also be provided in another way. For example, it is conceivable that the fibrous web sheet is guided over an even, heated surface while a further, unheated element, for example, a belt, exerts a controlled pressure.

It is preferably provided that the contact time between a portion of the fibrous web sheet and the heated surface amounts to between 0.05 s and 0.4 s. In order to adjust this contact time or, generally, the wrap angle of the fibrous web sheet on the heating roll, the consolidated nonwoven web is subsequently guided over a further guide roll. The position between the heating roll and the guide roll is decisive for the wrap angle and therefore also for the contact time as well as for the required pressure. In particular, it can be provided according to a preferred embodiment that the position between the two guide rolls is adjustable, particularly controllable, so that the angle of wrap at the heating roll with the heated surface can be varied. In this way, a wide variety of nonwoven webs can be produced and the required material characteristics can be adapted.

Further, in order to be able to press the fibrous web sheet against the heated surface to a sufficient extent, it is preferably provided that the fibrous web sheet is additionally consolidated in a further step, and this additional consolidation step is carried out prior to the thermal smoothing. In this regard, the fibrous web is still considered as such even after an additional consolidation step has been incorporated and the fibrous web has been reinforced to a certain extent. It is not considered a nonwoven web within the meaning of the invention until after the thermal smoothing.

Thermal calendering or through-air bonding, for example, can be provided as additional consolidating step. In thermal calendering, the fibrous web sheet is guided under pressure between two heated calender rolls, at least one of these calender rolls having a surface texture which forms fastening points and therefore an embossed pattern at discrete locations on the fibrous web sheet. The embossed pattern is formed by the discrete points comprising highly consolidated regions and regions which are not consolidated or only somewhat consolidated. Within the framework of the invention, the proportion of consolidated regions is preferably between 5% and 25% of the surface area. On the one hand, this ensures a sufficient consolidation; on the other hand, it is ensured that the asymmetrical effect with two different sides is maintained. Too high a proportion would destroy the softness of the unironed side.

In through-air bonding, the nonwoven web is guided through an oven and is heated uniformly by means of hot air. This hot air results in a partial fusing of the staple fibers of at least the first group and a thermal bonding of the fibers with one another.

Both types of consolidation are distinctly different from the reinforcement step of thermal smoothing. In thermal smoothing, an asymmetrical densification is carried out by guiding the nonwoven web along the heated surface exclusively by one side. In through-air bonding, by contrast, hot air circulates through the fiber layer which is accordingly substantially uniformly heated so that there is also no pronounced asymmetry with respect to the consolidation of the two sides. Further, compared to thermal calendering, the required pressure is much lower and the contact time is much longer. In through-air bonding, heating does not take place by means of contact heat but by means of the uniform circulation of hot air.

Accordingly, the nonwoven element which is produced as a result is preferably formed via two separate consolidation methods. The nonwoven is reinforced in its entirety by means of preconsolidation by through-air bonding or thermal calendering, while the thermal consolidation through the action of contact heat leads to an additional consolidation on one side of the nonwoven web, while the other side remains substantially unaffected. When more than one consolidation method is provided, the following sequences of consolidation steps have turned out to be particularly preferable:

1) calendering, thermal smoothing, through-air bonding

2) calendering, thermal smoothing

3) calendering, through-air bonding, thermal smoothing

4) through-air bonding, thermal smoothing

Further, the effect of the one-sided thermal smoothing can be reinforced in that the fibrous web sheet is cooled at least prior to or during the consolidation of the fibrous web sheet. For example, the first side can be guided past the heated surface of a guide roll and the second side can be guided past a cooled side of a cooling roller for this purpose. Alternatively, other measures, e.g., air cooling, which actively cool the second side of the fibrous web sheet can also be provided while the contact heat acts on the second side. Further, a pressure-exerting element, e.g., a cooled roll or a belt, can be introduced to amplify the smoothing effect on the heated side.

As has already been mentioned, apart from the contact time and the required pressure, the heating temperature of the heated surface also plays a significant role. This heating temperature amounts to between 120 and 200° C., particularly preferably between 140 and 180° C. according to a preferred embodiment.

According to a further development of the invention, the first side of the fibrous web sheet can also be heated in multiple stages in which a plurality of, e.g., two, three or more, heated surfaces are then preferably arranged one after the other in production direction. For example, this can involve a plurality of heating rolls. Starting from such an embodiment, the heated surfaces can have the same temperature so that the contact time is prolonged. However, an embodiment in which the heated surfaces have different temperatures is preferred, e.g., the temperature can increase in production direction.

The staple fibers of the first group are formed from a thermoplastic material which can fuse under the process conditions mentioned above. The staple fibers of the first group are preferably formed at least partially from a polyolefin. For example, the staple fibers may be formed exclusively from polyethylene (PE) or polypropylene (PP). Alternatively, the staple fibers can also be formed at least partially from biodegradable thermoplastic polymers, e.g., polylactide (PLA) or polyhydroxylalkanoates (PHA). Mixtures of different thermoplastic materials, particularly of the previously mentioned materials, are also contemplated.

Alternatively, staple fibers in the form of bicomponent fibers having, for example, a core of polypropylene or polyethylene terephthalate (PET) and a cladding of polyethylene (PE) can also be provided.

Further, the staple fibers can also be formed with non-round cross sections (as shaped fibers). Because of the non-round cross section, more fibers can be used per square meter. The visual appearance can also be a particular advantage. The staple fibers with a non-round cross section can be, for example, trilobal fibers which are formed either from polypropylene (PP) or from a combination of polyethylene (PE) and polypropylene.

Further, by using staple fibers, it is also possible to supply staple fibers from at least a second group formed from a non-fusible material to form the fibrous web sheet. Within the meaning of the invention, non-fusible materials are generally materials which are not formed from a thermoplastic material. However, materials having a melting temperature which is higher by at least 10 K, preferably by 20 K, than the heating temperature of the heated surface are also designated as non-fusible. Correspondingly, they can also be polymeric materials such as polyethylene terephthalate, for example. However, the second group of staple fibers is preferably formed at least in part but preferably completely from natural fibers. These natural fibers are selected in particular from the group comprising cotton, wool, silk, linen and hemp. The non-fusible materials are then fed to the individual consolidation steps together with the first group of fusible fibers and, after the melting of the staple fibers of the first group, bond with the latter so that a kind of matrix of molten staple fibers of the first group and staple fibers of the second group arranged in the matrix results after cooling.

Further, the staple fibers are carded before consolidation. Carding is a process by which the loose staple fibers are aligned to form a fibrous web. This takes place at the beginning in a carding machine.

Within the framework of the invention, it is sufficient in principle that only a first layer of staple fibers is consolidated to form a nonwoven web according to the methods mentioned above. However, it also lies within the scope of the invention to supply at least a second layer comprising a carded web prior to or subsequent to carding. This can also take place before or after a consolidation step, e.g., in the form of thermal smoothing. Even more pronounced differences between the different sides can be generated through the use of different layers of carded web. In this respect, it is also conceivable to supply a layer of carded web which is already preconsolidated by through-air bonding or calendering. This can be carried out, for example, before calendering or also after the thermal smoothing of the first layer. Further, the nonwoven web can also be connected to a substrate. This substrate can be a film, in particular a plastic sheet, for example.

At the conclusion, the individual nonwoven elements can be severed from the nonwoven web.

A further object of the invention is a nonwoven element for hygiene products which is obtainable in particular according to the method according to the invention with at least a first layer of staple fibers which extends in a longitudinal direction, a width direction running transverse to the latter and a thickness direction running perpendicular to the latter, the staple fibers being partially fused together proceeding from a first side. The staple fibers on the opposite second side are not fused together or are only fused together to a lesser extent than on the first side.

Accordingly, in contrast to the solutions known thus far, different properties, in particular haptic properties, can be made possible on both sides of the nonwoven element with only one layer of staple fibers. Accordingly, it is generally not necessary to connect different layers of different types of fiber to one another by means of an additional connecting step.

It is preferably provided that the density decreases along the thickness direction proceeding from the first side. Accordingly, the nonwoven element according to the invention is characterized especially by different material characteristics along the thickness direction. Proceeding from the first side, there results a relatively stiff and high-tensile portion adjoined by a further portion adjoining the second side of the nonwoven web, this further portion being distinguished by a particularly soft quality. Further, depending on the embodiment, the first side can be formed much smoother than the second side. This is useful particularly when the nonwoven element is to be glued against a further surface. The adhesive can then be applied to a large surface. At the same time, as a result of the consolidation on the first side, the adhesive cannot penetrate excessively into the nonwoven element and lead to a bonding inside of the nonwoven element.

Further, the nonwoven element according to the invention is distinguished by a low mass per unit area which is preferably between 10 and 60 g/m², particularly between 20 and 50 g/m². It is further noted in this regard that basically all of the material features of the method described above also apply to the nonwoven element according to the invention and vice versa.

The invention is further directed to a composite of the nonwoven element according to the invention and at least a further layer. The at least one further layer is preferably a carded web and/or films, particularly plastic films. The further carded web layers can be preconsolidated, e.g., by through-air bonding or by thermal calendering. The nonwoven element may be lined with the film, for example, to form the composite.

This also applies to a hygiene element according to the invention which is at least partially formed from a nonwoven element according to the invention. This hygiene element can be selected from the group comprising diapers, hygiene wet wipes and incontinence articles. According to a particularly preferred embodiment, this can be the outer chassis or the landing zone of a diaper.

### BRIEF DESCRIPTION OF THE DRAWINGS

The invention will be described in more detail in the following referring to an embodiment example. In the drawings:

FIGS. 1, 1A show a schematic diagram of the method according to the invention;

FIGS. 2, 2A show a variant of the method according to FIG. 1;

FIGS. 3, 3A show a further variant of the method according to FIG. 1;

FIG. 4 show an alternative adjustment possibility for the consolidation;

FIG. 5 show a nonwoven element according to the invention; and

FIG. 6 show an alternative embodiment of the nonwoven element according to FIG. 5.

### DETAILED DESCRIPTION OF THE EMBODIMENTS

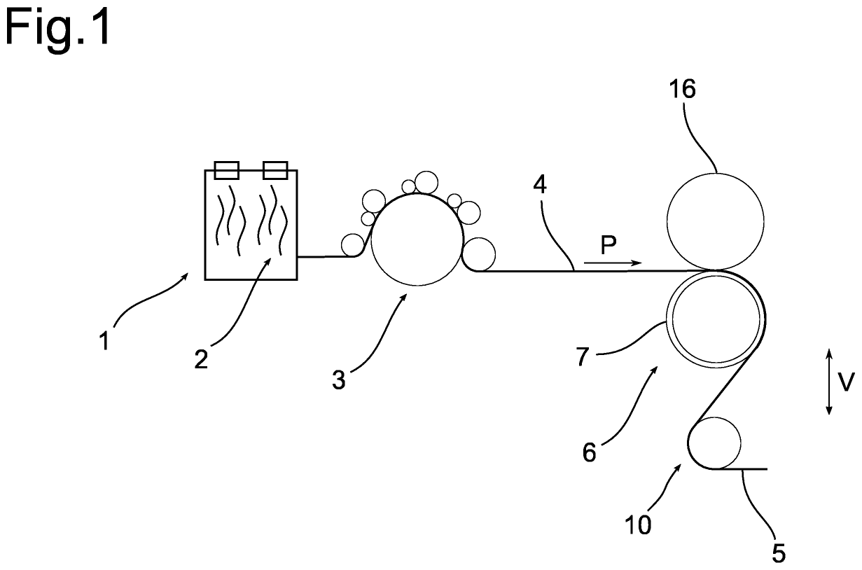

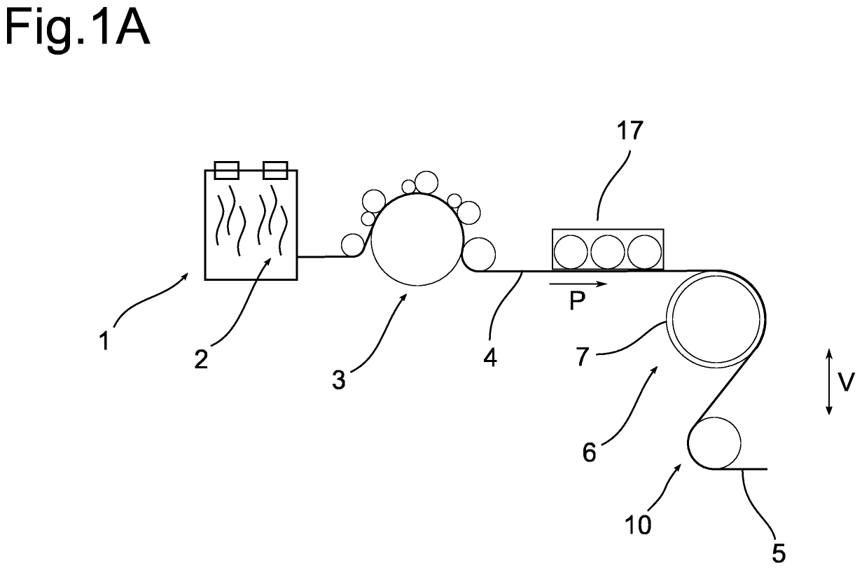

FIG. 1 schematically shows the method according to the invention. In a first work step, staple fibers of at least a first group 1 and of a second group 2 are fed to a carding machine 3 in which the staple fibers 1, 2 are aligned via a plurality of carding rolls and formed as a fibrous web sheet 4.

The fibrous web sheet 4 is guided in production direction P and extends in a width direction, not shown in more detail in FIG. 1, and in a thickness direction D perpendicular thereto. Accordingly, the fibrous web sheet 4 is formed exclusively from staple fibers 1, 2 and therefore requires subsequent consolidation to form a nonwoven web 5.

To this end, two separate consolidation steps are provided. A preconsolidation is carried out in the form of a thermal calendering. For this purpose, the fibrous web sheet 4 is guided between a calendering roll 16 and a heating roll 6. The fibrous web sheet 4 is compacted through the gap formed between the calendering roll 16 and the heating roll 6. Further, the calendering roll 16 has a surface structure such that the fibrous web sheet 4 is correspondingly compacted only in discrete locations. At the same time, heat is transferred to the fibrous web sheet 4 via the calendering roll 16 so that the fibrous web sheet 4 correspondingly melts in the compacted locations and accordingly connects the individual staple fibers 1, 2 to one another. The calendering roll 16 can also be dispensed with in principle. In that case, the fibrous web sheet 4 must be stabilized before being fed to the heating roll 6, for example, via a one-sided thermal preconsolidation so that the fibrous web sheet 4 does not disintegrate over the course of the thermal smoothing. Referring to FIG. 1A, this takes place by means of a roll arrangement 17 which acts on the horizontally running fibrous web sheet 4.

With this in mind, the staple fibers of the first group 1 are formed from a fusible material. The staple fibers of the second group 2, on the other hand, are formed from a non-fusible material, preferably natural fibers, e.g., cotton, wool, silk, linen, hemp or fibers of regenerated cellulose.

Consequently, the heating roll 6 forms a kind of counter-pressure element for the calendering roll 16. Moreover, a further consolidation step is carried out downstream of the first consolidation step via the heating roll 6. The heating roll 6 is a steel roll which is heated in the manner described above. The guide roll 6 has a heated surface 7 along which the fibrous web sheet 4 is guided. The fibrous web sheet 4 wraps around the heating roll 6 with a wrap angle α, the contact surface area between the fibrous web sheet 4 and the guide roll 6 increasing as angle α increases. Thus it will be seen that the fibrous web sheet 4 is guided past the heating roll 6 exclusively via a first side 8 and is heated through contact with the heated surface 7 such that the staple fibers of the first group 1 are partially fused. Since the second side 9 of the fibrous web sheet 4 is heated and, beyond this, the fibrous web sheet 4 is pressed against the heating roll 6 via the first side 8, a varying material density results along the thickness direction D in the nonwoven web 5 formed therefrom.

The extent to which the fibrous web sheet 4 is consolidated through contact with the heated surface 7 of the heating roll 6 depends substantially on the web velocity in production direction P, the contact surface area between the heated surface 7 and the fibrous web sheet 4, and on the web tension. The contact surface area is in turn decisively determined by the wrap angle α. Further, this wrap angle α is also decisive for the pressure between the heated surface 7 and the fibrous web sheet 4. In order to adjust this, a further guide roll 10 is provided and is formed in the depicted example to be displaceable in direction V to the heating roll 6. Alternatively, it is also possible, of course, to provide a horizontal displacement. In both cases, the wrap angle α is influenced. In the depicted example, the wrap angle α increases with decreasing distance so that the contact surface area between the fibrous web sheet 4 and the heated surface 7 is increased. Consequently, when the distance between the heating roll 6 and the guide roll 10 increases, the wrap angle α decreases and the contact surface area becomes smaller. On the whole, it is provided that the time during which a portion of the fibrous web sheet 4 is in contact with the heated surface 7 is between 0.05 s and 0.4 s based on the web velocity and the wrap angle α. The temperature of the heated surface 7 which is also decisive for the stiffening of the fibrous web sheet 4 is between 120 and 200° C.

Further, in the depicted example, bicomponent fibers comprising a core of polyethylene terephthalate and a cladding of polyethylene are used as staple fibers of the first group 1. The staple fibers of the second group 2 are cotton fibers. The exact composition of the utilized staple fibers will be apparent from Table 1.

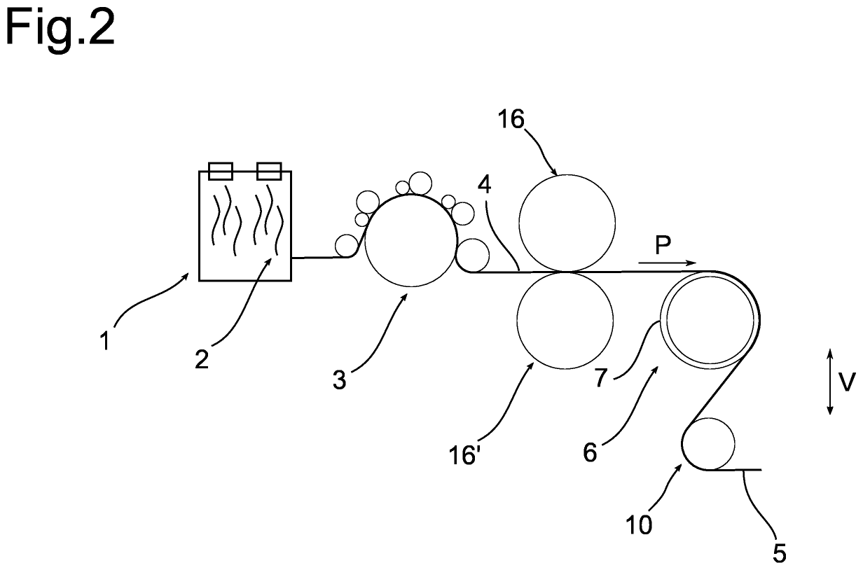

The method shown in FIG. 2 is largely identical to the method according to FIG. 1 except that two separate calendering rolls 16, 16′ are now provided for preconsolidation and the heating roll 6 provided for the actual consolidation is not a component part of the calendering device. As will be apparent from Table 1, the staple fibers of the first group 1 are staple fibers of polypropylene. The staple fibers of the second group 2 are again staple fibers of cotton.

TABLE 1

| | Staple fibers | Staple fibers | | Basis |
|---|---|---|---|---|
| Example | Group 1 | Group 2 | Mixture | weight |
| FIG. 1 | polypropylene (PP) | cotton length: | 85% PP + | 30 g/m² |
| | length: 35-45 mm | 24-28 mm; | 15% cotton | |
| | linear density: | micronaire | | |
| | 2.2 dtex | value: 4 | | |
| | melting point: | | | |
| | 158-161° C. | | | |
| FIG. 2 | bicomponent fiber | cotton length: | 85% BiCo + | 35 g/m² |
| | (BiCo): | 24-28 mm; | 15% cotton | |
| | PET/PE (core/ | micronaire | | |
| | cladding) length: | value: 4 | | |
| | 35-45 mm | | | |
| | linear density: | | | |
| | 1.7 dtex (PET) | | | |
| | 2.2 dtex (PE) | | | |
| | melting point | | | |
| | cladding: | | | |
| | 130-133° C. | | | |

With the method according to FIGS. 1 and 2, tests were carried out with the process parameters listed in Table 2. Examples Ia to Ic refer to a process flow according to FIG. 1. Examples IIa to IIc refer to a process flow according to FIG. 2.

TABLE 2

| Example | T_{cal} | | A_{emb} | P_{cal} | Tₕₑₐₜ | tₕₑₐₜ | | lₕₑₐₜ | | v_{web} | |
|---|---|---|---|---|---|---|---|---|---|---|---|
| Ia | 165/157° | C. | 20% | 110-125 | — | 0 | s | 0 | mm | 10 | m/min |
| | | | | N/mm | | | | | | | |
| Ib | 165/157° | C. | 20% | 110-125 | 150° C. | 1.26 | s | 209 | mm | 10 | m/min |
| | | | | N/mm | | | | | | | |
| Ic | 165/157° | C. | 20% | 110-125 | 160° C. | 1.26 | s | 209 | mm | 10 | m/min |
| | | | | N/mm | | | | | | | |
| IIa | 171° | C. | 10% | 110-125 | 161° C. | 0.01 | s | 3 | mm | 140 | m/min |
| | | | | N/mm | | | | | | | |
| IIb | 145° | C. | 10% | 110-125 | 157° C. | 0.10 | s | 24 | mm | 140 | m/min |
| | | | | N/mm | | | | | | | |
| IIc | 145° | C. | 10% | 110-125 | 157° C. | 0.24 | s | 24 | mm | 60 | m/min |
| | | | | N/mm | | | | | | | |

T_{cal}: temperature of the calender roll 16
A_{emb}: proportion of regions compacted over the course of calendering
P_{cal}: calendering pressure
Tₕₑₐₜ: temperature of the heating roll 6
tₕₑₐₜ: contact time with the heating roll 6
lₕₑₐₜ: contact length of the heating roll 6
v_{web}: velocity of the nonwoven web 5

Based on these process parameters, various nonwoven elements 14 were formed and underwent various quality tests. The results of these tests are shown in Table 3.

TABLE 3

| | MDT | MDE | CDT | CDT20 | CDE | MAR | MDBL |
|---|---|---|---|---|---|---|---|
| Example | [N/5 cm] | [%] | [N/5 cm] | [N/5 cm] | [%] | [degree] | [mN*cm] |
| Ia | 35.6 | 35.5 | 5.9 | 2.55 | 74 | 2 | 0.8 |
| Ib | 40.6 | 26.7 | 6.2 | 4.35 | 68 | 1 | 1.34 |
| Ic | 46.8 | 26.2 | 7.7 | 8.00 | 43 | 1 | 2.31 |
| IIa | 18.9 | 15.4 | 1.6 | 1.07 | 59 | 5 | 0.61 |
| IIb | 22.3 | 11.7 | 2.2 | 1.73 | 37 | 3.5 | 0.86 |
| IIc | 40.4 | 16.8 | 4.1 | 3.23 | 47 | 2 | 2.18 |

where,
MDT = tensile strength in machine direction
MDE = elongation at break in machine direction
CDT = tensile strength in transverse direction
CDT20 = tensile strength in transverse direction at 20% elongation
CDE = elongation at break in transverse direction
MAR = Martindale test
MDBL = bending length in machine direction

The values for tensile strength and elongation at break were determined in accordance with EN ISO 13934-1:1999, and the bending length was determined in accordance with EN ISO 9073-7:1998. The MAR value is a measurement of abrasion resistance and pilling tendency. The determination is made by measurement using the Martindale method in accordance with ASTM D4966-98 and WSP 20.5(05). Wear of material is measured by subjecting the respective product to rubbing motion in the form of a geometric figure. The abrasion resistance is evaluated with a grade by then comparing the material to the known visual standard under the stated criteria. The lower the grade, the better the abrasion behavior of the nonwoven. Within the framework of the invention, a grade of at least 2 is aimed for. The criteria for grading are listed in the following Table 4:

TABLE 4

| Grade | Acceptable | Criteria |
|---|---|---|
| 5 | − | pilling or cords form network of a plurality of |
| | | individual fibers and a loft >10 mm |
| | | hole formation >10 mm |
| | | large portion of the sample is worn away |
| 4 | | pilling or cords form network of a plurality of |
| | | individual fibers and a loft >5 mm |
| 3 | | formation of a kind of yarn from long twisted |
| | | fibers >2 mm |
| | | height of yarn <5 mm |
| | | no network formation |
| 2 | + | pill formation diameter <2 mm |
| | | thread formation with a width <2 mm |
| | | no network formation |
| 1 | | short fibers are raised |
| | | slight pilling with diameter <2 mm |

Further, it is decisive for the quality of the nonwoven element 14 that the CDT20 value is especially high. A certain compromise must always be made between wear resistance and the softness of the material. Trials have shown that the quality increases with an increasing contact time and also at higher temperatures of the heating roll 6.

The method according to FIG. 2A substantially conforms to the method according to FIG. 2, but now a cooled steel roll 11 is provided which cools the second surface 9 of the fibrous web sheet 4 at the start of consolidation, while the first side 8 of the fibrous web sheet 4 is heated via the heated surface 7 of the heating roll 6. In this way, it is ensured that the staple fibers on the second side 9 of the fibrous web sheet 4 remain substantially unaffected by the introduction of heat through the heating roll 6. Further, instead of a cooled roll 11, another kind of cooling mechanism can be provided. For example, the second surface 9 may be acted upon by cooled air.

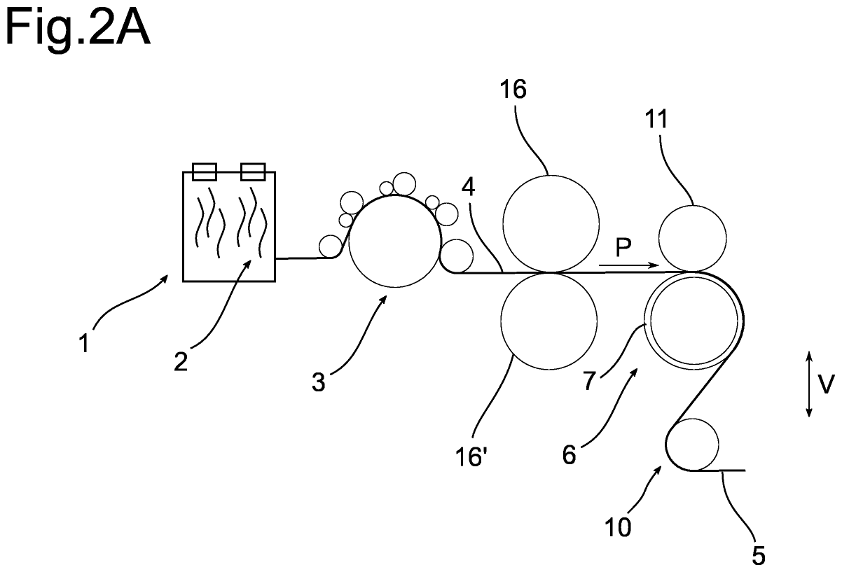

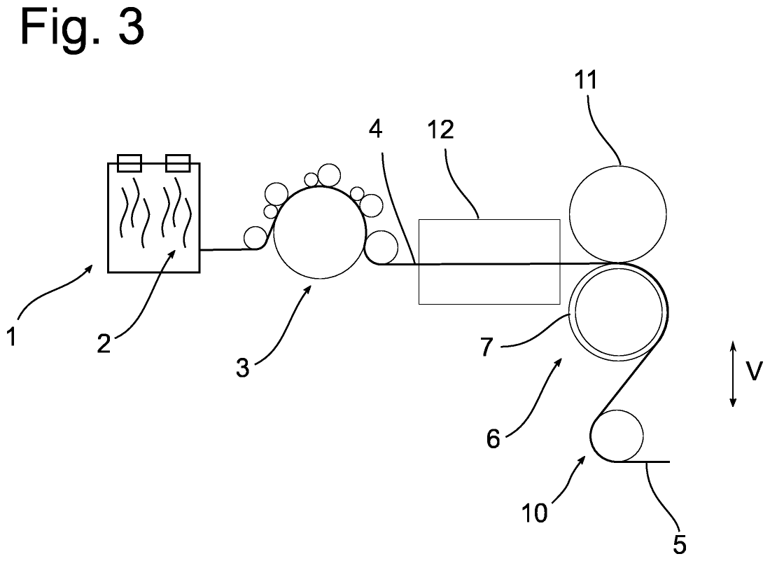

In the method according to FIG. 3, an oven 12 is provided instead of calendering rolls 16, 16′, the fibrous web sheet 4 running through this oven 12. The fibrous web sheet 4 is acted upon by hot air in the oven 12 so that the staple fibers of the first group 1 are partially fused and bond with one another and with the staple fibers of the second group 2 homogeneously over the thickness D. Only then is the fibrous web sheet 4 which is consolidated in this way fed to the heating roll 6 having the heated surface 7 and additionally consolidated on one side. According to FIG. 3A, the oven can also first be arranged after the heating roll 6.

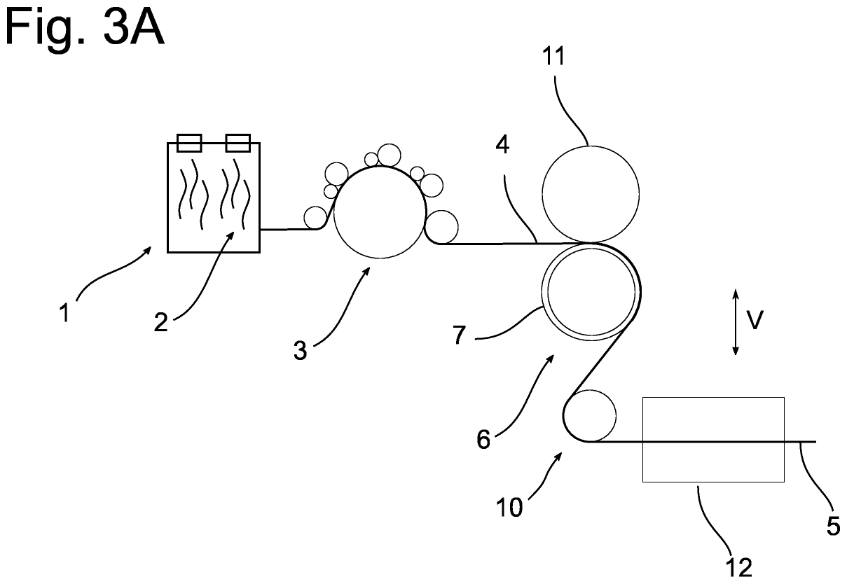

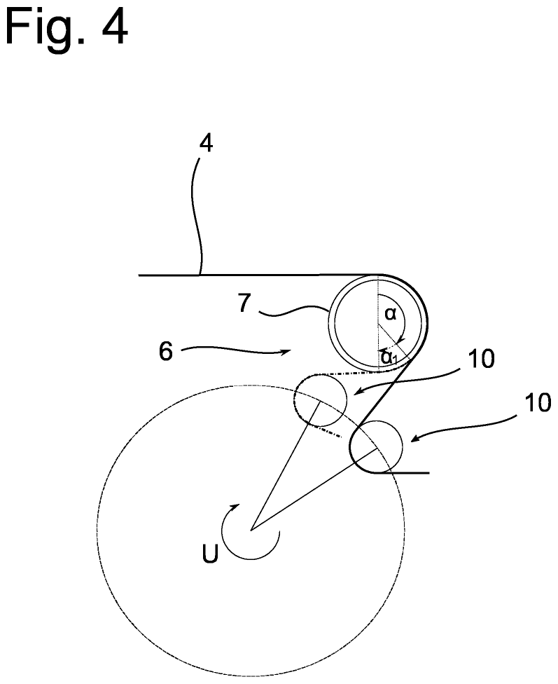

FIG. 4 shows an alternative possibility for adjusting the heating roll 6 and the guide roll 10 relative to one another. This adjusting possibility is crucial for the wrap angle α. As angle α increases, the contact surface between the fibrous web sheet 4 and the heated surface 7 is also increased. According to the adjusting possibility in FIGS. 1 to 3, the guide roll 10 is moved linearly in direction of the heating roll 6 for this purpose. However, in the embodiment according to FIG. 4 the guide roll 10 is rotatable along a circular path 13, and the wrap angle α increases in rotational direction D. The guide roll 10 is shown in a total of two different positions in FIG. 4. The wrap angle α according to the first position is smaller than wrap angle α₁ according to the second position of the guide roll 10. In the second position, the wrap angle α₁ amounts to approximately 180° so that the heated surface 7 rotates halfway corresponding to the fibrous web sheet 4 and a contact time between the fibrous web sheet 4 and the heated surface 7 is much longer than in the first position of the wrap angle α with the web velocity remaining the same.

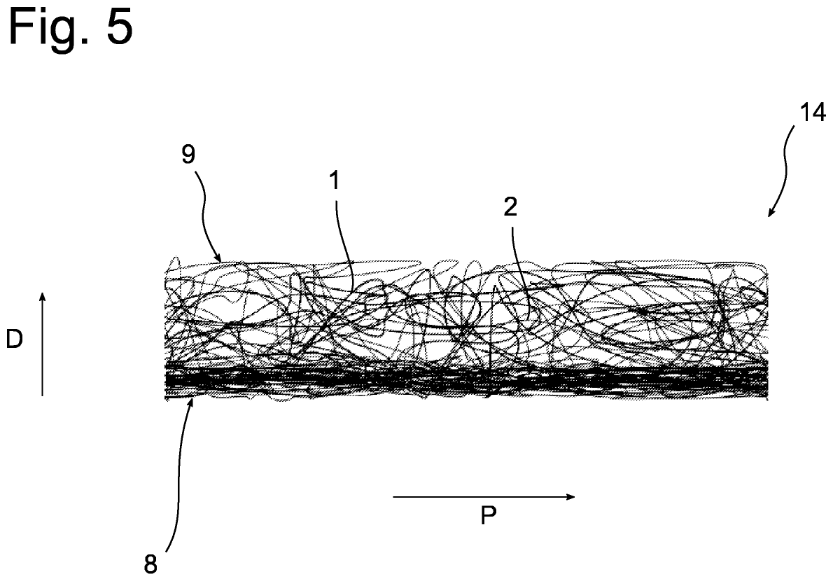

FIG. 5 shows a nonwoven element 14 which is formed from the nonwoven web 5 and severed therefrom. The nonwoven element 14 extends over a length running in production direction P, a width which is not shown in more detail and a thickness extending perpendicular thereto. It can be clearly seen that there are different densities on the two sides 8, 9 of the nonwoven element 14 as a result of treating with the hot surface 7. While the side 8 directly contacting the heated surface 7 was very highly compacted, the second side 9 is hardly influenced, if at all, by the consolidation step. Accordingly, the density decreases in direction of the second surface 9 proceeding from the first surface 8. This drop in density is not necessarily continuous but rather approximately stepwise.

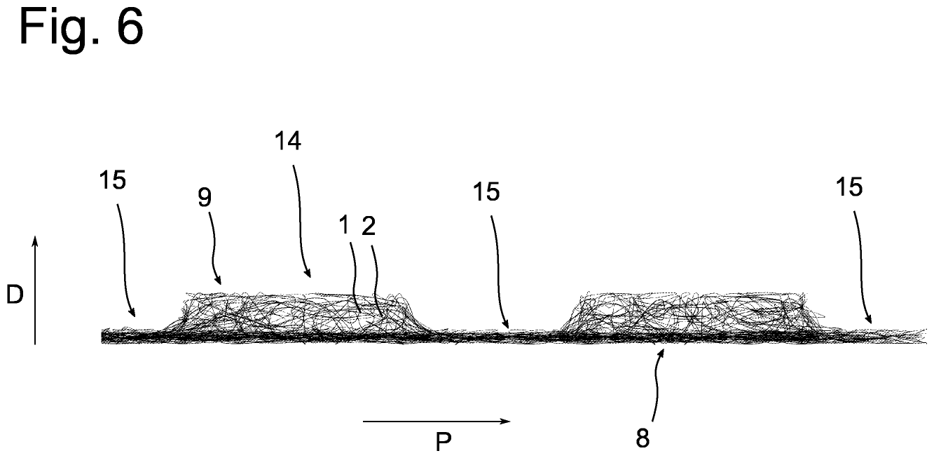

FIG. 6 shows a nonwoven element 14 according to FIG. 5 in which regions 15 are additionally provided by thermal calendering at discrete points at which the nonwoven element 14 has been completely compacted and consolidated and correspondingly has a much smaller thickness than in regions which have not been thermally calendered. Such an embodiment may be useful in order to additionally consolidate the nonwoven element 14. By combining the two consolidation methods, the quantity of thermally calendered regions 15 can be significantly reduced compared to consolidation exclusively by thermal calendering. It should be noted here that these points are always weakened points in the material which should be kept as small as possible depending on the embodiment of the nonwoven element 14.

What is claimed is:

1. A method for producing a nonwoven element for hygiene products, comprising at least the following steps:
forming a fibrous web sheet with a width direction extending transverse to a production direction and a thickness direction perpendicular to the width direction by supplying staple fibers from at least a first group which are formed from a thermoplastic material,
consolidating the fibrous web sheet to form a nonwoven web by heating exclusively a first side of the fibrous web sheet through contact with a heated surface such that the staple fibers of the first group are partially melted, and
cooling the nonwoven web.

2. The method according to claim 1, wherein the heated surface is part of a heating roll over which the fibrous web sheet is guided.

3. The method according to claim 2, wherein a contact time between a portion of the fibrous web sheet and the heated surface amounts to between 0.05 s and 0.4 s.

4. The method according to claim 1, wherein the heated surface has a heating temperature of between 120 and 250° C.

5. The method according to claim 1, wherein the staple fibers of the first group are formed at least partially from polyolefin.

6. The method according to claim 1, wherein the staple fibers of the first group are formed at least partially from biodegradable thermoplastic polymers.

7. The method according to claim 1, wherein a second side of the fibrous web sheet is partially cooled.

8. The method according to claim 1, wherein at least staple fibers from a second group formed from a non-fusible material are supplied to form the fibrous web sheet.

9. The method according to claim 8, wherein the staple fibers of the second group are natural fibers selected from the group consisting of cotton, wool, silk, linen, hemp and fibers of regenerated cellulose.

10. The method according to claim 1, wherein the staple fibers are carded to form the fibrous web sheet.

11. The method according to claim 1, wherein through-air bonding or a thermal calendering is used to consolidate the fibrous web sheet.

12. The method according to claim 11, wherein the heating roll is formed as a calender roll of a calendering device.

13. The method according to claim 1, wherein at least a further layer is supplied, the further layer being selected from the group consisting of staple fibers, carded web and film.

14. The method according to claim 1, further comprising the step of severing individual nonwoven elements from the nonwoven web, after said step of cooling.

15. A nonwoven element for hygiene products, obtained according to the method of claim 1, wherein the element comprises a layer of staple fibers which extends in a longitudinal direction, a width direction running transverse to the longitudinal direction and a thickness direction running perpendicular to the width direction, and wherein the staple fibers are partially fused together proceeding from a first side.

16. The nonwoven element according to claim 15, wherein a density decreases along the thickness proceeding from the first side.

17. The nonwoven element according to claim 15, wherein the element has a mass per unit area between 10 and 60 g/m².

18. A hygiene element which is at least partially formed from a nonwoven element according to claim 15.

19. The hygiene element according to claim 18, which is selected from the group consisting of diapers, hygiene wet wipes and incontinence articles.
